(19) United States
(12) Patent Application Publication
(10) Pub. No.: US 2007/0203425 A1
(43) Pub. Date: Aug. 30, 2007

(54) Apparatus And Method For Detecting Anomalies Within Body

(75) Inventors: Eung Je Woo, Seongnam-si (KR); Jin Keun Seo, Seoul (KR); Oh In Kwon, Seoul (KR)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: UNIVERSITY-INDUSTRY COOPERATION GROUP OF KYUNGHEE UNIVERSITY, Yongin, Kyungki-do (KR)

(21) Appl. No.: 11/568,950

(22) PCT Filed: Apr. 27, 2005
(86) PCT No.: PCT/KR05/01216

§ 371 Date: Nov. 10, 2006

(30) Foreign Application Priority Data
May 13, 2004 (KR) 10-2004-0033928

(51) Int. Cl. A61B 5/05

(52) U.S. Cl. 600/547

(57) ABSTRACT

Apparatus and method for detecting a lesion, the method including the steps of applying a voltage to an electrode in contact with a part of a body, sensing signals from an examining part of the body by using probe (14) having a plurality of sensing electrodes (143), and calculating a position and a size of a lesion in the body with reference to the signals sensed thus, whereby detecting not only existence of a lesion, but also a position and a size of the lesion.

### TECHNICAL FIELD

The present invention relates to apparatuses and methods for detecting a lesion, such as cancer or tumor, by using electric properties of a body.

### BACKGROUND ART

According to the annual report of the central cancer registry, it can be noted that there is ceaseless increase of breast cancers and colorectum cancers in the case of women. Particularly, the incidence rate of breast cancers has increased continuously to become a first place (16.1%) of women cancer incidence rates in 2001, and it is foreseen that it will increase continuously. In a case of Japan, the breast cancer incidence rate was 19.6% of all cancer incidence rates in 2001, and in a case of the USA, the breast cancer incidence rate was 37.3% of all cancer incidence rates in 2001.

The most preferable treatment is early examination, and treatment of the tumor before the tumor transfers to other part of the body. However, such an early diagnosis requires a positive and effective diagnosis method. Presently, efforts for the early diagnosis of the breast cancer are under progress actively by using various methods. For the early diagnosis, a variety of medical apparatus is used, such as apparatus of X-ray mammography, ultrasonography, thermography, MRI, CT, and so on. However, in the case of the X-ray mammography which is used the most widely, due to characteristics of the X-ray, the X-ray mammography has a disadvantage in that the diagnosis of young women who has a high density of a lobule-alveolar-system is difficult, and, furthermore, there is a hazard of the breast cancer caused by the X-ray used for the diagnosis. In the case of the ultrasonography, the ultrasonography has a disadvantage in that resolution is low, such that there are ceaseless discussion on the effectiveness of the ultrasonography, and application to the early diagnosis is difficult. In the case of the thermography which utilizes a point that cancerous tissue or inflammation has a temperature higher than a normal cell, it is difficult to diagnosis the breast cancer by using the thermography, because the thermography shows an image of temperatures of skin, mainly. MRI or CT requires high diagnosis cost and is difficult to take an image of the breast only.

Recently, there have been researches for detecting the breast cancer by using a phenomenon in which there is a three to ten times difference of conductivities between a normal tissue and a tumor tissue. Of the researches, T-Scan of the TransScan Medical Ltd. takes a method of application of a voltage and measurement of current, to present a transfer admittance map of the organic tissue. The measuring method of the T-Scan is identical to the frontal plane impedance camera which is the first electrical impedance tomography (EIT) of which research is started from early 1980s. That is, an electrostatic voltage is applied to a part of human body by using a surface electrode, currents from array electrodes attached to the other part of the human body are measured while the voltage of the array electrodes are maintained at the ground potential, and the measured currents are displayed as an image. The image is called as a transfer admittance image. The T-Scan presents the transfer admittance image, and the user determines a patient having a breast cancer by making subjective interpretation of the image. In other words, the diagnosis apparatus like the T-Scan fails to be provided with a function for analyzing data measured on a surface of the breast. Therefore, it is difficult for the T-Scan to make an accurate detection of a lesion because a diagnosis result is dependent on a visual determination of the user.

### DISCLOSURE OF INVENTION

An object of the present invention is to provide apparatus and method for detecting a lesion, which can make accurate detection of, not only existence of lesion like a breast cancer, but also a size of the lesion with reference to a transfer admittance data measured at a surface of a human body.

The object of the present invention can be achieved by providing an apparatus for detecting a lesion including an electrode for applying a voltage to a part of a body, a probe having a plurality of sensing electrodes for sensing signals from an examining part of the body, and a control unit for determining a position and a size of a lesion in the body with reference to the signals sensed by the sensing electrodes.

The probe includes a plate for arranging the sensing electrodes at regular intervals thereon, and a metal frame around the plate for maintaining the sensing electrodes at a reference voltage.

The control unit determines a pinpoint of the lesion in a probe plane opposite to a center of the lesion with reference to the sensed signals, and calculates a distance from the center of the lesion to the pinpoint of the lesion in the probe plane for determining a position of the lesion. The control unit calculates a volume of the lesion with reference to an average of the sensed signals, a distance between the center of the lesion and the pinpoint of the lesion in the probe plane, an intensity of the signal sensed at the pinpoint of the lesion in the probe plane, for determining a size of the lesion.

In another aspect of the present invention, there is a provided method for detecting a lesion including the steps of applying a voltage to an electrode in contact with a part of a body, sensing signals from an examining part of the body by using probe having a plurality of sensing electrodes, and calculating a position and a size of a lesion in the body with reference to the signals sensed thus.

The step of applying a voltage includes the step of applying two or more voltages of different frequencies to the electrode in succession.

The step of sensing signals includes the step of the probe sensing currents flowing through the examining part.

The step of calculating a position and a size of a lesion includes the step of determining a pinpoint of the lesion in a probe plane opposite to a center of the lesion with reference to the sensed signals, calculating a distance between the center of the lesion and the pinpoint of the lesion in the probe plane, and calculating a volume of the lesion.

There are two kinds of steps for determining a pinpoint of the lesion in a probe plane opposite to a position of a center of the lesion with reference to the sensed signals. The first kind of step includes the steps of calculating a difference between an intensity of the sensed signal and a reference intensity for each of the sensing electrodes, and determining a position of the sensing electrode having a maximum value of the differences as the pinpoint of the lesion in the probe plane opposite to the center of lesion. The second kind of step includes the steps of calculating an intensity difference between a signal of the first frequency and a signal of the second frequency of the sensed signals for each of the sensing electrodes, and determining a position of the sensing electrode having a maximum value of the differences as the pinpoint of the lesion in the probe plane opposite to the center of lesion.

Also, there are two kinds of steps for calculating a distance between the center of lesion and the pinpoint of the lesion in the probe plane. The first kind of step includes the step of calculating a distance between the center of the lesion and the pinpoint of the lesion in the probe plane with reference to a distance between the pinpoint of the lesion in the probe plane and any point in the probe plane, an intensity of the signal sensed at the pinpoint of the lesion in the probe plane, and an intensity of the signal sensed at the any point in the probe plane. The second kind of step includes the step of calculating a distance between the center of the lesion and the pinpoint of the lesion in the probe plane with reference to a distance between the pinpoint of the lesion in the probe plane and any point in the probe plane, an intensity of the signal of the first frequency and an intensity of the signal of the second frequency sensed at the pinpoint of the lesion in the probe plane, and an intensity of the signal of the first frequency and an intensity of the signal of the second frequency sensed at the any point in the probe plane.

The step of calculating a volume of the lesion includes the step of calculating a volume of the lesion with reference to an average of intensities of the sensed signals, a distance between the center of the lesion and the pinpoint of the lesion in the probe plane, and an intensity of the signal sensed at the pinpoint of the lesion in the probe plane, or alternatively, includes the step of calculating a volume of the lesion with reference to an average of intensities of the sensed signals of the first frequency, a distance between the center of the lesion and the pinpoint of the lesion in the probe plane, and an intensity of the signal of the first frequency and an intensity of the signal of the second frequency sensed at the pinpoint of the lesion in the probe plane.

### BRIEF DESCRIPTION OF DRAWINGS

FIG. 1 illustrates a block diagram of an apparatus for detecting a lesion in accordance with a preferred embodiment of the present invention;

FIG. 2 illustrates a diagram showing a method for detecting a lesion by using an apparatus for detecting a lesion in accordance with a preferred embodiment of the present invention;

FIG. 3 illustrates a block diagram of the electrostatic voltage generator in FIG. 1;

FIG. 4 illustrates a diagram of a scan probe of the present invention;

FIGS. 5A, 5B, and 5C illustrate diagrams each showing a variation of a scan probe of the present invention;

FIG. 6 illustrates a block diagram of the current measuring unit in FIG. 1;

FIG. 7 illustrate diagrams showing images of measured parts by using the device for detecting lesion of the present invention.

### BEST MODE FOR CARRYING OUT THE INVENTION

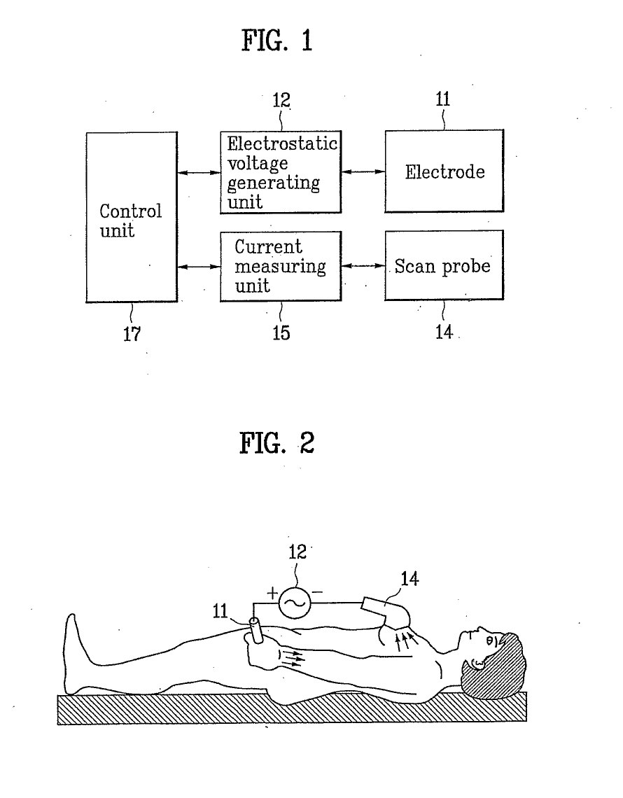

Preferred embodiments of the present invention will be described in detail with reference to the attached drawings. FIG. 1 illustrates a block diagram of an apparatus for detecting a lesion in accordance with a preferred embodiment of the present invention, and FIG. 2 illustrates a diagram showing a method for detecting a lesion by using an apparatus for detecting a lesion in accordance with a preferred embodiment of the present invention.

There is an electrode 11 in contact with a part of a body for applying a voltage to the body. As shown in FIG. 2, the electrode 11 provides the voltage to an inner part of the body through a part of the body, such as a hand.

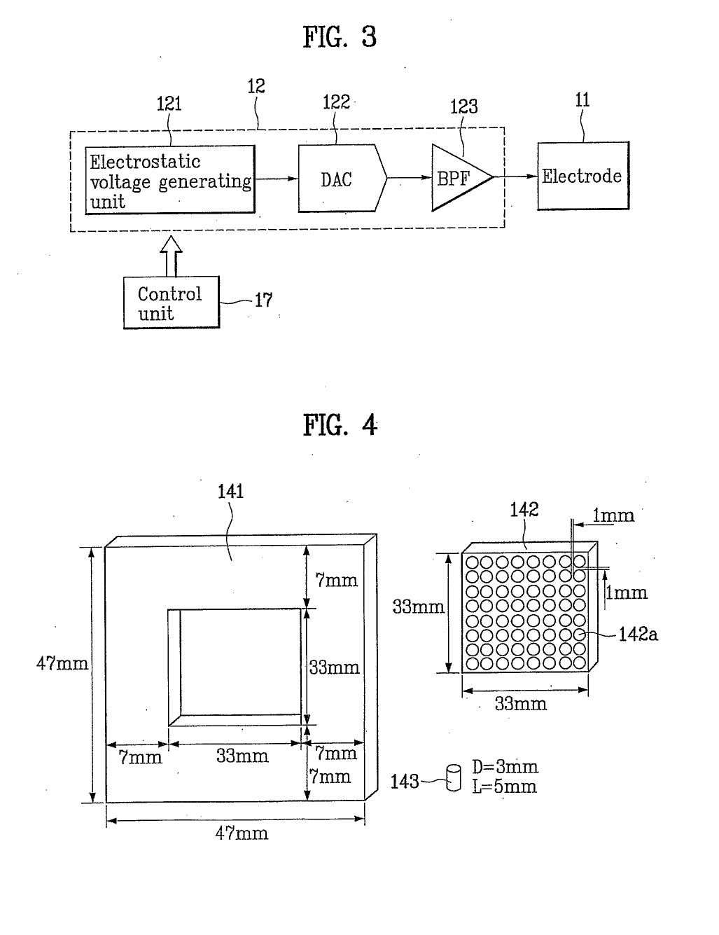

The voltage generating unit 12 supplies the voltage to the electrode 11. The voltage generating unit 12 generates one or more than one particular voltages of sinusoidal waves having frequencies different from each other, and provides to the electrode 11. As shown in FIG. 3, the voltage generating unit 12 includes an FPGA (field programmable gate array) based digital waveform generating unit 121, a digital-analog converter (DAC) 122, and a band pass filter (BPF) 123.

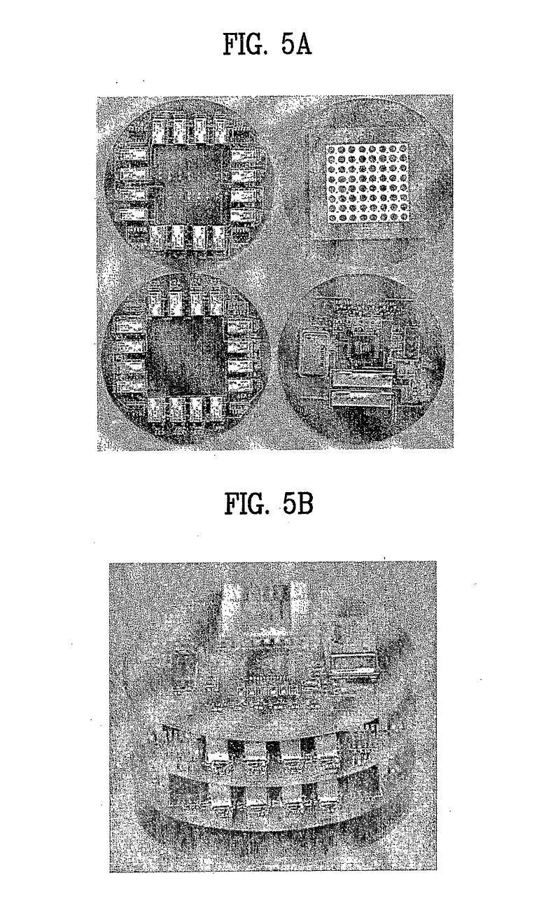

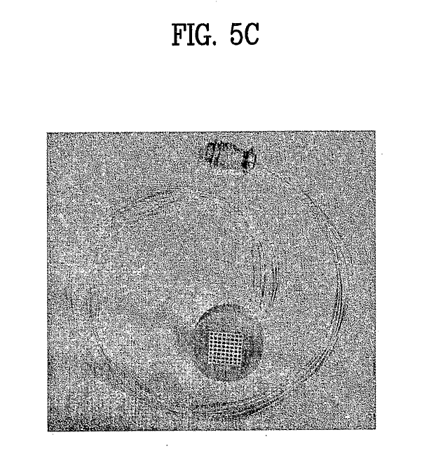

The scan probe 14 is brought into contact with a part of body intended to examine, and senses signals flowing through the part intended to examine, i.e., currents. If it is intended to detect breast cancer, the scan probe 14 scans currents at a breast. The scan probe 14 includes planar array of electrodes. The planar array of electrodes sense the currents flowing through the part intended to examine in a state a reference voltage thereof is maintained. FIG. 4 illustrates a scan probe of the present invention. As shown in FIG. 4, the scan probe 14 includes an array of cylindrical sense electrodes 143 arranged at fixed intervals. For an example, the scan probe 14 includes 8×8, or 16×16 electrodes 143. In order to arrange the electrodes 143 at fixed intervals, a plate 142 for fixedly securing the electrodes 143 is required. The plate 142 has holes 142a for placing the electrodes 143 therein, respectively, and is formed of an insulating material for electrically isolating the electrodes 143. The plate 142 has a metal frame 141 around the plate 142. The metal frame 141 maintains the electrodes 143 and neighborhood thereof at the reference voltage (0V). FIGS. 5A, 5B, and 5C illustrate diagrams each showing a variation of a scan probe of the present invention. The scan probe has sensing electrodes and switches joined together. As shown, the scan probe includes a substrate of the planar array of electrodes of a plurality of the sensing electrodes, a plurality of switch substrates, and a switch control substrate stacked together. The switch substrates and the switch control substrate enable the plurality of sensing electrodes to provide signals through a small number of cables.

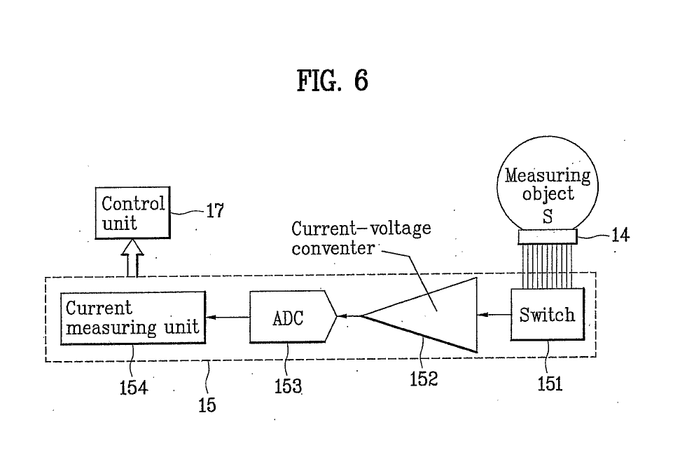

A current measuring unit 15 measures intensities of the currents sensed at the electrodes 143. As shown in FIG. 6, the current measuring unit 15 includes a switch 151 for providing the currents sensed at the electrodes 143 selectively, a current-voltage converter 152 for converting the current from the switch into a voltage, an analog-digital converter (ADC) 153 for converting an analog signal from the current-voltage converter 152 into a digital signal, and a digital phase sensitive current measuring unit 154 for receiving a signal from the analog-digital converter 153, and measuring intensities of fine-currents sensed at the electrodes 143 with reference to the signal.

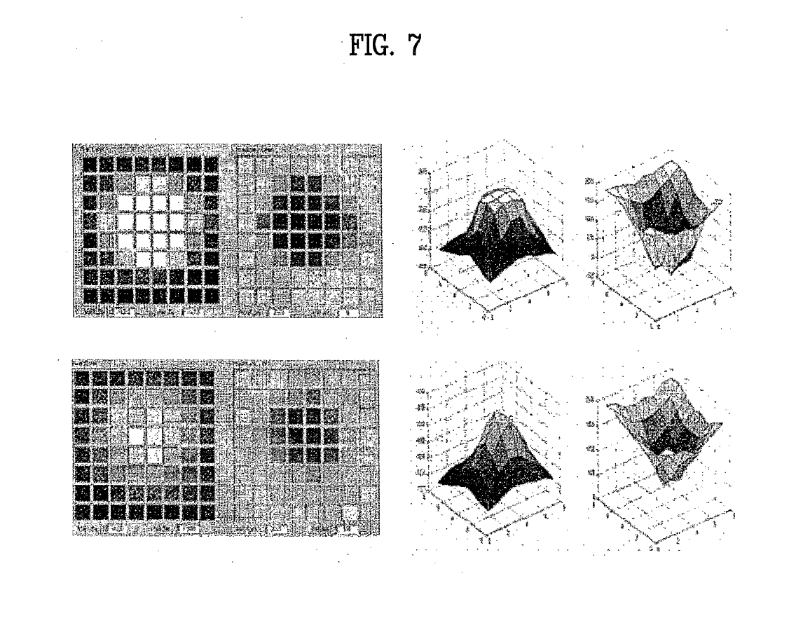

Data on the currents measured at the current measuring unit 154 is provided to the control unit 17, and the control unit 17 detects a real component (magnitude) and an imaginary component (phase) of each of the currents, and calculates a transfer admittance data based thereon. Since the current flows through a lesion, such as a cancer, or tumor tissue, better than a normal tissue, existence of the lesion can be determined by using the transfer admittance data that is proportional to the current intensities. The control unit 17 also can calculate a position and a size of the lesion by using the transfer admittance data, and display an inner part of the examined part in a 3-D image by using the calculated values as shown in FIG. 7. The image provides information on conductivity and permittivity of the organic tissue, such that the user knows, not only existence of cancer, tumor or the like, but also the position and size of the lesion, accurately.

Embodiments of a method for detecting the position and the size of the lesion with reference to the transfer admittance data will be described.

### FIRST EMBODIMENT

The control unit 17 calculates the position and the size of the lesion by using the following equation of an algorithm, and makes the lesion into an image by using the data on the position and the size of the lesion. The relation of the transfer admittance data, and the position and the size of the lesion can be defined as the following equation (1).

$$g ( x , y ) - {g}_{0} ( x , y ) = A \frac{6 α ( {τ}_{1} - {τ}_{2} )}{2 {τ}_{1} + {τ}_{2}} \frac{2 {d}^{2} - {( x - {ξ}_{1} )}^{2} - {( y - {ξ}_{2} )}^{2}}{4 {π [ {( x - {ξ}_{1} )}^{2} - {( y - {ξ}_{2} )}^{2} + {d}^{2} ]}^{5 / 2}} \qquad ( 1 )$$

where,

A denotes a volume of the lesion,

(x,y) denotes any point in a plane Γ of the scan probe 14,

g(x,y) denotes a distribution of measured transfer admittances (a function defined in the Γ),

α denotes an average of the transfer admittance distribution g(x,y) in the scan probe 14 plane Γ,

g₀(x,y) denotes the transfer admittance distribution when no lesion exists,

d denotes a vertical distance from a point (ξ₁, ξ₂) in the scan probe 14 plane to a center of gravity of the lesion,

(ξ₁, ξ₂) denotes a pinpoint in the scan probe 14 plane opposite to the lesion in the body,

τ₁=σ₁+jωξ₁, where σ₁ denotes an average conductivity of a normal tissue of breast, ξ₁ denotes an average permittivity of a normal tissue, and ω denotes a frequency of a sinusoidal wave,

τ₁=σ₂+jωξ₂, where σ₂ denotes an average conductivity of a normal tissue of breast, and ξ₂ denotes an average permittivity of a normal tissue.

The following equations (2)˜(4) can be derived from above equation (1), which enables to estimate the position and the size of the lesion with reference to the transfer admittance distribution.

$$( {ξ}_{1} , {ξ}_{2} ) = \begin{matrix}arg max \\ ( x , y ) ∈ Γ\end{matrix} \vert g ( x , y ) - {g}_{0} ( x , y ) \vert \qquad ( 2 )$$

The above equation (2) enables to obtain a pinpoint (ξ₁, ξ₂) in the scan probe 14 plane Γ opposite to the position of lesion (a central point of the lesion) in the body. The pinpoint (ξ₁, ξ₂) in the scan probe 14 plane Γ is a point in the scan probe 14 plane Γ at which an absolute value |g(x,y)−g₀(x,y)| is the maximum. In order to obtain the pinpoint (ξ₁, ξ₂), the control unit 17 compares the measured admittances g(x,y) to the reference admittances g₀(x,y), respectively. For an example, if the scan probe 14 has 8×8 sensing electrodes 143, the control unit 17 calculates differences of the admittance values measured at the 64 sensing electrodes 143, and reference admittance values for the 64 sensing electrodes 143, respectively. That is, a difference of a measured admittance value and the reference admittance value is calculated for each of the sensing electrodes 143. Then, one of the sensing electrodes 143 having a maximum value of the difference values is fixed as the pinpoint (41, 42) in the scan probe 14 plane opposite to the position of the lesion.

$$\vert \frac{g ( {ξ}_{1} , {ξ}_{2} ) - {g}_{0} ( {ξ}_{1} , {ξ}_{2} )}{g ( x , y ) - {g}_{0} ( x , y )} \vert = \frac{\vert 2 - \frac{{l}^{2}}{{d}^{2}} \vert}{2 {( \frac{{l}^{2}}{{d}^{2}} + 1 )}^{5 / 2}} \qquad ( 3 )$$

The equation (3) enables to obtain a depth of the lesion d. The (x,y) denotes any point in the scan probe 14 plane Γ in the neighborhood of the pinpoint (ξ₁, ξ₂), and λ denotes a distance between the point (x,y) and the pinpoint (ξ₁, ξ₂), λ=\sqrt{(x−ξ₁)²+(y−ξ₂)²}. If the pinpoint (ξ₁, ξ₂) in the scan probe 14 plane opposite to the lesion position is obtained from the equation (2), the control unit 17 calculates the distance X between the pinpoint (ξ₁, ξ₂), and the any oint (x,y). Then, the control unit 17 calculates a distance ‘d’ from the pinpoint (ξ₁, ξ₂) in the scan probe 14 plane to the lesion with reference to the measured admittance value g(ξ₁, ξ₂), and the reference admittances g₀(ξ₁, ξ₂) at the pinpoint (ξ₁, ξ₂), the measured admittance value g(x,y) and the reference admittances g₀(x,y) at the any point (x, y), and the λ. As can be noted in the equation (3), the distance ‘d’ is dependent on a ratio of a difference between two admittance values g(x,y)−g₀(x,y), and the a difference between the two admittance values g(ξ₁, ξ₂)−g₀(ξ₁, ξ₂), as well as λ. If the pinpoint (ξ₁, ξ₂) in the scan probe 14 plane opposite to the lesion position, and the distance d from the pinpoint (ξ₁, ξ₂) to the lesion are known, the position of the lesion in the body can be known, accurately.

$$A = \frac{π \vert 2 + \frac{{τ}_{2}}{{τ}_{1}} \vert {d}^{3} \vert g ( {ξ}_{1} , {ξ}_{2} ) - {g}_{0} ( {ξ}_{1} , {ξ}_{2} ) \vert}{3 \vert 1 - \frac{{τ}_{2}}{{τ}_{1}} \vert \vert α \vert} \qquad ( 4 )$$

Above equation (4) enables to obtain a size(volume) A of the lesion. The τ₂/τ₁ is a preset ratio of conductivities, or permittivties between the lesion and the normal tissue. Once the distance d from the pinpoint (ξ₁, ξ₂) in the scan probe 14 plane to the lesion is calculated, the control unit 17 can calculate the volume A of the lesion. As expressed in above equation (4), it can be noted that the volume A of the lesion is calculated with reference to the distance d, the difference g(ξ₁, ξ₂)−g₀(ξ₁, ξ₂) of the measured admittance value g(ξ₁, ξ₂), and the reference admittance value g₀(ξ₁, ξ₂), and the average value α of the transfer admittance distribution g(x,y) in the scan probe 14 plane.

If it is intended to estimate the position and the size of the lesion actually by using the equation, a value of the reference admittance distribution g₀(x,y) is required for a case no lesion exists. The value of the reference admittance distribution g₀(x,y) can be obtained by using any one of the following methods.

First, the admittance distribution value of a part symmetry to the part of the body intended to examine is used. For an example, if it is intended to examine a left breast, the admittance distribution measured at the right breast is used as the reference admittance distribution, and vice versa.

Second, an average of the measured admittance distribution g(x, y) is used as the reference admittance distribution g₀(x,y).

Third, a statistical value (average value) of the admittance distribution obtained through many experiment without lesions is used as the reference admittance distribution g₀(x,y). For an example, in a case if a woman in her 20s is examined for breast cancer, an average of the admittance distribution obtained from women of 20s without breast cancer is used as the reference admittance g₀(x,y).

### SECOND EMBODIMENT

In order to implement the second embodiment, the electrostatic voltage generating unit 12 generates electrostatic voltages sinusoidal waves having frequencies different from each other, and the electrodes 11 apply the two sinusoidal electrostatic voltages to the body in succession. After detecting transfer admittance distributions of the sinusoidal electrostatic voltages of difference frequencies through the scan probe 14, the control unit 17 calculates a size and a position of the lesion by using the detected transfer admittance distributions.

If the sinusoidal electrostatic voltages of different frequencies are used, it is assumed that the transfer admittance distributions g(x,y), and \tilde{g}(x,y) are for the two sinusoidal electrostatic voltages of different frequencies ω, and \tilde{ω}, and complex conductivities of a normal tissue and a tumor tissue for the frequency \tilde{ω} are \tilde{τ}₁=σ₁+j\tilde{ω}ξ₁, and \tilde{τ}₂=σ₂+j\tilde{ω}ξ₂ . Then, relation of the transfer admittance distributions for the two sinusoidal electrostatic voltages of different frequencies ω, and \tilde{ω} and the position and the size of the lesion can be defined as the following equation (5).

$$\tilde{g} ( x , y ) - g ( x , y ) = A \frac{18 α ( {τ}_{2} - {\tilde{τ}}_{2} )}{( 2 {τ}_{1} + {τ}_{2} ) ( 2 {\tilde{τ}}_{1} + {\tilde{τ}}_{2} )} \frac{2 {d}^{2} - {( x - {ξ}_{1} )}^{2} - {( y - {ξ}_{2} )}^{2}}{4 {π [ {( x - {ξ}_{1} )}^{2} - {( y - {ξ}_{2} )}^{2} + {d}^{2} ]}^{5 / 2}} \qquad ( 5 )$$

That is, the electrostatic voltages of different frequencies are applied, and one of the transfer admittance distributions for the electrostatic voltages of different frequencies is used as a reference admittance distribution. An algorithm for detecting the position and the size of the breast cancer with reference to the equation (5) is as follows.

$$( {ξ}_{1} , {ξ}_{2} ) = \begin{matrix}arg max \\ ( x , y ) ∈ Γ\end{matrix} \vert \tilde{g} ( x , y ) - {g}_{0} ( x , y ) \vert \qquad ( 6 )$$

The above equation (6) enables to obtain the pinpoint (ξ₁, ξ₂) in the scan probe 14 plane Γ opposite to the position of lesion. The pinpoint (ξ₁, ξ₂) in the scan probe 14 plane Γ is a point in the scan probe 14 plane Γ at which |\tilde{g}(x,y)−g(x,y)| is the maximum. In order to obtain the pinpoint (ξ₁, ξ₂), the control unit 17 compares the admittance distribution \tilde{g}(x,y) for \tilde{ω} to the admittance difference g(x,y) for ω. Then, a point in the scan probe 14 plane having a maximum value of the differences of the two admittances is fixed as the pinpoint (ξ₁, ξ₂) in the scan probe 14 plane opposite to the lesion position.

$$\vert \frac{g ( {ξ}_{1} , {ξ}_{2} ) - {g}_{0} ( {ξ}_{1} , {ξ}_{2} )}{g ( x , y ) - g ( x , y )} \vert = \frac{\vert 2 - \frac{{l}^{2}}{{d}^{2}} \vert}{2 {( \frac{{l}^{2}}{{d}^{2}} + 1 )}^{5 / 2}} \qquad ( 7 )$$

The equation (7) enables to obtain a depth of the lesion d. The (x, y) denotes any point in the scan probe 14 plane Γ in the neighborhood of the pinpoint (ξ₁, ξ₂), and λ denotes a distance between the any point (x, y) and the pinpoint (ξ₁, ξ₂), λ=\sqrt{(x−ξ₁)²+(y−ξ₂)²}. The control unit 17 calculates the distance λ between the any point (x, y) and the pinpoint (ξ₁, ξ₂), and calculates a distance d from the pinpoint (ξ₁, ξ₂) in the scan probe 14 plane to the lesion with reference to the two admittance values \tilde{g}(ξ₁, ξ₂), and g(ξ₁, ξ₂) measured at the pinpoint (ξ₁, ξ₂), the two admittance values \tilde{g}(x,y), g(x,y) measured at the any point (x, y), and the λ. As can be noted in the equation (7), the distance d is dependent on a ratio of a difference between two admittance values \tilde{g}(x,y)−g(x,y), and a difference between the two admittance values \tilde{g}(ξ₁, ξ₂)−g(ξ₁, ξ₂0, as well as λ. If the pinpoint (ξ₁, ξ₂) of the lesion in the scan probe 14 plane, and the distance d from the pinpoint (ξ₁, ξ₂) to the lesion are known, the position of the lesion in the body can be known, accurately.

$$A = \frac{π \vert 2 {τ}_{1} + {τ}_{2} \vert \vert 2 {\tilde{τ}}_{1} + {\tilde{τ}}_{2} \vert \tilde{g} ( {ξ}_{1} , {ξ}_{2} ) - g ( {ξ}_{1} , {ξ}_{2} ) \vert {d}^{3}}{9 \vert α \vert \vert {τ}_{2} - {\tilde{τ}}_{2} \vert} \qquad ( 8 )$$

Above equation (8) enables to obtain a size(volume) A of the lesion. Once the distance d from the pinpoint (ξ₁, ξ₂) in the scan probe 14 plane to the lesion is calculated, the control unit 17 can calculate the volume A of the lesion. As expressed in above equation (8), it can be noted that the volume A of the lesion is dependent on the distance d, the difference \tilde{g}(ξ₁, ξ₂)−g(ξ₁, ξ₂) of the two admittance values, and the average value α of the transfer admittance distribution \tilde{g}(x,y) in the scan probe 14 plane, as well as data τ₁, τ₂, \tilde{τ₁}, and \tilde{τ₂} on the conductivities and permittivities for the two sinusoidal electrostatic voltage.

### INDUSTRIAL APPLICABILITY

As has been described, the present invention permits, not only determination of existence of lesion in the body, but also a position and a size of the lesion with reference to admittance data. As a technology for detecting breast cancer, fabrication of an apparatus is possible, which costs lower, and harmless to a human body than the related art apparatus. Accordingly, the present invention can be useful in diagnosis of breast cancer.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method for detecting a lesion comprising the steps of:
applying a voltage to an electrode in contact with a part of a body;
sensing signals from an examining part of the body by using probe having a plurality of sensing electrodes; and
calculating a position and a size of a lesion in the body with reference to the signals sensed thus.

2. The method as claimed in claim 1, wherein the step of applying a voltage includes the step of applying two voltages of different frequencies to the electrode in succession.

3. The method as claimed in claim 1, wherein the step of sensing signals includes the step of the probe sensing currents flowing through the examining part.

4. The method as claimed in claim 1, wherein the step of calculating a position and a size of a lesion includes the step of;
determining a pinpoint of the lesion in a probe plane opposite to a center of the lesion with reference to the sensed signals,
calculating a distance between the center of the lesion and the pinpoint of the lesion in the probe plane, and
calculating a volume of the lesion.

5. The method as claimed in claim 4, wherein the step of determining a pinpoint includes the steps of;
calculating a difference between an intensity of the sensed signal and a reference intensity for each of the sensing electrodes, and
determining a position of the sensing electrode having a maximum value of the differences as the pinpoint of the lesion in the probe plane opposite to the center of lesion.

6. The method as claimed in claim 4, wherein the step of determining a pinpoint includes the steps of;
calculating an intensity difference between a signal of the first frequency and a signal of the second frequency of the sensed signals for each of the sensing electrodes, and
determining a position of the sensing electrode having a maximum value of the differences as the pinpoint of the lesion in the probe plane opposite to the center of lesion.

7. The method as claimed in claim 4, wherein the step of calculating a distance between the center of the lesion and the pinpoint of the lesion in the probe plane includes the step of calculating a distance between the center of the lesion and the-pinpoint of the lesion in the probe plane with reference to a distance between the pinpoint of the lesion in the probe plane and any point in the probe plane, an intensity of the signal sensed at the pinpoint of the lesion in the probe plane, and an intensity of the signal sensed at the any point in the probe plane.

8. The method as claimed in claim 4, wherein the step of calculating a distance between the center of the lesion and the pinpoint of the lesion in the probe plane includes the step of calculating a distance between the center of the lesion and the pinpoint of the lesion in the probe plane with reference to a distance between the pinpoint of the lesion in the probe plane and any point in the probe plane, an intensity of the signal of the first frequency and an intensity of the signal of the second frequency sensed at the pinpoint of the lesion in the probe plane, and an intensity of the signal of the first frequency and an intensity of the signal of the second frequency sensed at the any point in the probe plane.

9. The method as claimed in claim 4, wherein the step of calculating a volume of the lesion includes the step of;
calculating a volume of the lesion with reference to an average of intensities of the sensed signals, a distance between the center of the lesion and the pinpoint of the lesion in the probe plane, and an intensity of the signal sensed at the pinpoint of the lesion in the probe plane.

10. The method as claimed in claim 4, wherein the step of calculating a volume of the lesion includes the step of;
calculating a volume of the lesion with reference to an average of intensities of the sensed signals of the first frequency, a distance between the center of the lesion and the pinpoint of the lesion in the probe plane, and an intensity of the signal of the first frequency and an intensity of the signal of the second frequency sensed at the pinpoint of the lesion in the probe plane.

11. An apparatus for detecting a lesion comprising:
an electrode for applying a voltage to a part of a body;
a probe having a plurality of sensing electrodes for sensing signals from an examining part of the body; and
a control unit for determining a position and a size of a lesion in the body with reference to the signals sensed by the sensing electrodes.

12. The apparatus as claimed in claim 11, wherein the probe includes;
a plate for arranging the sensing electrodes at regular intervals thereon, and
a metal frame around the plate for maintaining the sensing electrodes at a reference voltage.

13. The apparatus as claimed in claim 11, wherein the control unit determines a pinpoint of the lesion in a probe plane opposite to a center of the lesion with reference to the sensed signals, and calculates a distance from the center of the lesion to the pinpoint of the lesion in the probe plane for determining a position of the lesion.

14. The apparatus as claimed in claim 13, wherein the control unit calculates a difference value between an intensity of each of the sensed signals of the sensing electrodes and a reference intensity, and determines a position of the sensing electrode which has a maximum value of the difference values as the pinpoint in the probe plane opposite to the center of lesion.

15. The apparatus as claimed in claim 13, wherein the control unit calculates a difference value between an intensity of a signal of the first frequency and an intensity of a signal of the second frequency of the sensed signals of the sensing electrodes, and determines a position of the sensing electrode which has a maximum value of the difference values as the pinpoint in the probe plane opposite to the center of lesion.

16. The apparatus as claimed in claim 13, wherein the control unit calculates a distance between the center of the lesion and the pinpoint of the lesion in the probe plane with reference to a distance between the pinpoint of the lesion in the probe plane and any point in the probe plane, the intensity of the signal sensed at the pinpoint of the lesion in the probe plane, and the intensity of the signal sensed at the any point of the lesion in the probe plane.

17. The apparatus as claimed in claim 11, wherein the control unit calculates a volume of the lesion with reference to an average of the sensed signals, a distance between the center of the lesion and the pinpoint of the lesion in the probe plane, an intensity of the signal sensed at the pinpoint of the lesion in the probe plane, for determining a size of the lesion.

18. The apparatus as claimed in claim 11, further comprising an electrostatic voltage generating unit for providing electrostatic voltages of sinusoidal waves having frequencies different from each other.
